(19) United States
(12) Patent Application Publication
(10) Pub. No.: US 2013/0190882 A1
(43) Pub. Date: Jul. 25, 2013

(54) Humeral Head Prosthesis

(71) Applicant: Shoulder Options, Inc., Waxhaw, NC (US)

(72) Inventor: C. Scott Humphrey, Eagle, ID (US)

(73) Assignee: SHOULDER OPTIONS, INC., Waxhaw, NC (US)

(21) Appl. No.: 13/745,844

(22) Filed: Jan. 20, 2013

Related U.S. Application Data
(60) Provisional application No. 61/589,243, filed on Jan. 20, 2012.

(51) Int. Cl. A61F 2/40

(52) U.S. Cl. CPC A61F 2/4014
USPC 623/19.14

(57) ABSTRACT

A humeral head prosthetic device including a chassis and a head attached or attachable to the chassis. In the preferred embodiment, the chassis includes a tapered and multifaceted anchor element attached to and projecting distally from a base element, the configuration of the tapered and multifaceted anchor element adapted to counter rotation of the chassis once it is impacted into the humeral head. The preferred embodiment of humeral head prosthetic device also includes one or more blind holes each of the blind holes or slots being defined in part by a penetrable wall, each of the blind holes including a wall that may be readily penetrated in order permit insertion of a tool to aid in removal of the prosthesis where such removal is required.

This application claims the benefit of the filing date of a prior-filed Provisional Application Ser. No. 61/589,243 entitled Humeral Head Prosthesis, filed Jan. 20, 2012, which is incorporated by reference herein.

### BACKGROUND OF THE INVENTION

1. Field of the Invention

The present invention relates to devices and methods for repairing the shoulder joint and more specifically to shoulder prostheses and methods of performing a shoulder arthroplasty.

2. Background

Until relatively recently, a total repair of the shoulder joint, or shoulder arthroplasty, required the removal of the entire head of the humerus bone, commonly at or below the anatomic neck, in order to accommodate the insertion of an elongated shaft of the device into the diaphysis of the bone. An articulation surface attached or attachable to the proximal end of the shaft and cooperates with a second articulation surface attached to the boney portion of the scapula. A relatively recent advance in total shoulder repair has been the introduction of the humeral head prosthesis, a device that mounts or attaches to a prepared surface of the humeral head. These devices also include a joint head having an articulation surface typically formed as a spherical segment configured to articulate against a cooperating surface attached to the boney portion of the scapula. The joint head is mounted to a mounting fixture which, according to the present state of the art, includes a variety of configurations. The mounting fixture is attached to the humeral head at a site located substantially at and coplanar to a distal margin of the anatomical head that has been modified by sawing, shaping and/or drilling to accommodate attachment of the mounting fixture to the modified humeral head.

In U.S. Published Patent Application 2009/0292364 by Linares, a shoulder joint implant assembly is described that includes a plastic ball secured to a first bone end face and a plastic receiver secured to a second bone end face. A fastener is secured to the first bone end face and over which is mounted the plastic ball. The receiver is anchored by a pair of inwardly extending mounting tabs that extend into cavities machined in the first bone.

U.S. Pat. No. 7,648,530 to Habermeyer, et al., describes a humeral head prosthesis including a joint head including a nearly spherical joint surface. A hollow screw fixes a mounting disk to a planed surface of the bone. The hollow screw is driven into the bone through a medial hole located in a collar formed on the proximal surface of the mounting disk. A cone shaped receiver is formed within the joint head and is suitably configured to receive the collar in a press fit.

U.S. Pat. No. 7,611,539 to Bouttens, et al., describes a shoulder prosthesis that includes a mounting plate having a central tubular mounting bushing. A joint head including an articulation surface formed as a spherical segment having an axial screw is fit over the mounting plate and the axial screw is screwed into the tubular mounting bushing.

U.S. Pat. No. 7,608,109 to Dalla Pria, describes a first articulation element associated with the top of the humerus that includes a two piece support member the first piece including a threaded stud for insertion into the humeral head, a second piece having a post that inserts into the core of the stud, the second piece also including several tabs that extend over the surface of the proximal end of the bone with screws that attach to the bone through the tabs. A joint head including an articulation surface formed as a spherical segment is attachable to the support structure.

In U.S. Pat. No. 6,969,406 to Tornier, a multi-piece shoulder prosthesis is described that has at least four principal assembled components including:
a) an anchor member having a tubular anchor extending from a base element,
b) a screw having a first external thread for threadedly engaging an internal thread located in the tubular anchor,
c) a bushing having an internal thread configured to threadedly engage a second external thread formed on the screw, and
d) a joint head including an articulation surface formed as a semi-spherical segment that includes a recess formed on an underside or the joint head. The recess includes an internal thread for engaging an external thread of the bushing. The recess of the joint head is configured such that as the internal thread of the joint head is advanced against the external thread of the bushing an inner surface of the recess seats against an outer annular surface of the base element.

While the devices and methods discussed above resolve some of the challenges related to providing a humeral head prosthesis, still a need exists to provide increasingly simplified humeral head prosthetic devices and systems. Additionally, a need exists to refine and improve upon the means by which the prosthesis is located on and secured to a prepared site located on the proximal humerus bone. In addition there is a need to provide a prosthetic device and system that includes improved means and features that allow for easier removal of the device in cases where such removal is indicated. Another issue left unaddressed by the current state of the art is the osteolysis that tends to occur in regions where there is no growth of the bone into the implant. Additionally, osteolysis is known to occur at the calcar region in patients who receive a prosthetic shoulder that includes a shaft that extends into the diaphysis of the bone. Other improvements may be seen in a humeral head prosthetic device by the addition of means for attaching a suture to the prosthesis so that the prosthesis may be utilized as an anchor for sutures that are used to re-attach connective tissue to the bone following installation of the prosthesis.

Therefore one object of the present invention is to provide a humeral head resurfacing prosthetic device. Another object of the present invention is to provide a prosthetic device and system that includes improved means and features that allow for easier removal of the device in cases where such removal is indicated. Yet another object of the present invention is to provide a modular system for repair of the humeral head that includes a variety of device anchors having a range of diameters and lengths and a variety of prosthetic heads having a range of head sizes and diameters. Another object of the present invention is to provide an improved methodology for locating the humeral head prosthetic device with respect to a modified humeral head. Another object of the present invention is to provide a prosthetic device for a long bone that reduces the tendency of osteolysis that has been observed in connection with the use of some prosthetic implants. Another object of the present invention is to provide a humeral head prosthetic device that includes one or more apertures through which a suture may be passed, the apertures adapted and optimized to facilitate the use of sutures for augmenting soft tissue connection. An additional object of the present invention is to provide a humeral head prosthetic device that has been optimized for removal in the event that such removal is indicated.

### SUMMARY OF THE INVENTION

The present invention is directed to a humeral head prosthetic device includes a chassis having a base element from which a tapered and multifaceted anchor element projects distally. The tapered and multifaceted anchor element is fixed to or formed upon or attachable to an articulation surface. In the preferred embodiment the humeral head prosthetic device the articulation surface is formed as a semi-spherical segment that is removably attachable to the chassis.

In one embodiment the humeral head prosthetic device is configured as a unitary prosthesis wherein the articulation surface is permanently fixed to or formed at the proximal end of the chassis and a contact surface includes a configuration that is suitable for mounting against a prepared surface of the humeral head. In one embodiment of the unitary prosthesis, the tapered and multifaceted anchor element is fixed or formed against an inner concave surface of the semi-spherical segment such that the inner concave surface becomes the contact surface and the outer convex surface serves as the articulation surface. This embodiment is intended for resurfacing, as opposed to replacing, the humeral head. In another embodiment, the joint head is separable from the chassis. Whether the prosthetic device is configured for resurfacing the joint or replacing the humeral head, the device includes a multifaceted anchor element fixed to or formed on the distal surface of the articulation surface. As for the unitary humeral head prosthetic device, the advantages of this configuration lie in part in its unitary construction and therefore the potential that it has to reduce costs associated with shoulder arthroplasty hardware and procedures.

In the embodiment wherein the joint head is separable from the chassis, the joint head is held against the base element by means of a cooperating press fit between a tapered post and socket. In one embodiment the post and socket each include a cooperating Morse taper. One advantage of this configuration lies in allowing the surgeon to select a chassis of having a size that is most appropriate for the size of the humeral head being repaired and a head including an articulation surface that cooperates best with the patient's glenoid. Another advantage of this configuration lies in creating an option of placing auxiliary anchors through the base element into the humeral head which results in a more stable placement of the prosthesis in the bone, particularly immediately post surgery, before boney in-growth between the bone and the central stem member has had time to progress. Another advantage of this configuration lies in creating an option of replacement of the joint head where a condition allowing for such a repair presents itself

The tapered and multifaceted anchor element of the humeral head prosthesis is defined by an inner wall surface and an outer wall surface. The wall of the anchor element has a tapering cross-section that diminishes in thickness as the distance from the base element increases, (distally converging). The surface of the inner wall of the anchor element is relatively featureless. The surface of the outer wall of the anchor element includes a series interconnected fins, each fin comprising a pair of converging wall segments or facets that taper distally as a distance from the base element increases, (distally converging). Apertures are formed through the wall of the anchor element to promote bone growth and revascularization. In alternate embodiments, the tapered and multifaceted anchor element may be formed as a single tubular element or it may be formed as a plurality of tubular segments.

In a preferred embodiment, the invention, the converging wall segments formed on the outer wall of the anchor element converge or truncate at a distance proximal to a distal lip of the anchor element. A series of circular or oval apertures are formed through the wall of the anchor element just proximal to the distal lip. These apertures are provided to accommodate a method of the invention discussed herein below. In order to prevent surface abrasion against sutures that may be guided through the apertures, the region of the wall of the anchor element just proximal to the distal lip through which these apertures are formed may include a polished finish with the annular edges of the apertures being radiused and polished as well.

At least a portion of both the inner wall and the outer wall of the region of the anchor element proximal to the polished region, as well as the contact surface of the base element exhibit a roughened surface. For instance the surfaces may be coated with a porous coating adapted to encourage bone in-growth, and hence enhanced mechanical properties at the site of impaction. Alternately, surfaces may be roughened by a process, such as grit blasting or machining, for example.

In one embodiment, the humeral head prosthetic device includes a plurality of anchors that are adapted for placement through and engagement with the base element of the chassis. Preferably, two anchors are set into the greater tuberosity and one anchor is set into the calcar region. Once the anchors are set, and due to that fact that the three anchors diverge from one another and the primary axis of the device, the placement of the device with respect to the bone has become remarkably stable resisting both pull-out and rotational forces. It is particularly important that any humeral head prosthetic device remain stable and have good resistance to pull out particularly just following implantation and before the bony in-growth that follows surgery has occurred.

Several characteristics of bony anatomy are addressed by the configuration of the humeral head prosthetic device of the present invention. First, the head of a bone is composed of an open-celled latticed material called trabecular bone encased in a sheath of cortical bone. Trabecular bone is adapted to absorb loads at the joint. In furtherance of this adaptation, trabecular bone exhibits a latticed structure that gives it a sponge-like appearance. As an individual ages, the size of a cavernous or osteopenic region of the bone increases from the inside towards the outside of the bone, decreasing the mass and density of the trabecular bone.

A second observation about bony anatomy is stated by Wolff's law which observes that where loading on bone increases, the bone remodels itself over time to become stronger in order to resist the load. Of particular note, and consistent with Wolff's law, is the observation that internal architecture of the trabecular bone undergoes adaptive change when subjected to stress. Wolff's law also recognizes that where loading on a bone decreases, the bone will become weaker. This phenomenon has been identified at least in part for the occurrence of osteolysis at the calcar region of the humerus in patients who receive a prosthetic shoulder that includes a shaft that extends into the diaphysis of the bone, thereby relieving much of the normal stress seen at this region due simply to anatomical configuration and load bearing at the joint.

The configuration of the tapered and multifaceted anchor element of the present invention and the preferred method of implanting anchor is directed at avoiding these physical limitations of aging bony anatomy by maximizing anchor to bone surface area in order to take advantage of impacting the device in a manner that promotes bony in-growth between the anchor member and the trabecular bone. The interconnected fins formed on the outer wall of the anchor element project out and away, further towards the periphery, and further from the center of the bone. The combined configurations of the tapering cross-section of the wall of the anchor element and the configuration of the proximally diverging outer wall segments or facets against the substantially vertical walls of the circular cut made in the prepared end of the humerus create substantial hoop stresses between the anchor element and the matrix of exposed trabecular bone into which the anchor element is impacted, which creates stresses at the site of impaction which in turn stimulates bony in-growth and remodeling over time in order to resist the load. According to a preferred methodology of implanting the anchor member, the interior of the anchor member may be packed with bone graft material to accelerate and promote boney in-growth. Additionally, bone growth is stimulated where it comes in contact with the roughened surfaces of the anchor element and the entire contact surface of the base element. As such, once in-growth has progressed at the bone-to-prosthesis interface, the occurrence of osteolysis promoted by mechanical stress shielding or introduction of foreign matter such as polyethylene debris at the bone-to-prosthesis interface should reduce substantially.

In the case where removal is required, several structural adaptations have been incorporated in the design of the humeral head prosthetic device of the present invention. For example, while a substantial portion of the anchor member of the prosthesis of the present invention includes a roughened surface or a surface coated with a porous coating, the porous surface extends only a portion of the length of the anchor member, so that bony growth is not promoted the full length of the anchor member towards the distal end of the prosthesis.

Additionally, and in furtherance of the objective of facilitating removal of an implantable prosthesis in the event that removal is required, one or more of “blind” holes or slots may be formed in a surface of the implantable prosthesis that would be used to aid in the removal of the chassis from the bone once impacted. Each blind hole or slot includes a penetrable wall that in part defines the blind hole or slot. In a case where removal of an implanted prosthesis is indicated, a drill or osteotome may be used to penetrate the penetrable wall formed at the bottom of each “blind” hole or slot. The drill or osteotome may then continue through the bone to help separate the prosthesis from the bone in a controlled manner. A thin metal layer or a frangible wall composed of cement, for instance a polymethylmethacrolate cement, or plastic would serve as a barrier with the purpose of keeping foreign material, particularly polyethylene debris, from finding its way through the “blind” hole into the bone over time. The surface of the wall section that is oriented towards the bone would preferably include a roughened surface consistent with the surrounding surfaces of the base element to promote bony in-growth against the surface of the base element. Alternately a “blind” hole may be created simply by filling a portion of a hole or slot formed in the prosthesis with a cement or plastic thereby forming a penetrable wall which bony growth may readily occur.

In a preferred embodiment, the device is to be manufactured and offered in a variety of sizes. In particular the diameter at the base of the joint head and the diameter of the base element are offered in increments through a range of diameters so that the device size can be matched as closely as reasonably possible to a particular patient's bone. Preferably, the diameter of the base element is the same as the diameter of the prepared site of the proximal humerus bone such that the base element's roughened surface contacts both the trabecular bone as well as the more dense cortical bone, thereby preventing subsidence of the device in the softer trabecular bone. The in-growth of the bone to the prosthesis that occurs at the periphery of the prepared bone should also serve as a barrier with the purpose of keeping foreign material, particularly polyethylene debris, from finding its way into the bone over time.

Preferably, the device is manufactured having dimensions within the following ranges:

Head diameter=35-55 mm

Head height=12-28 mm

Anchor length=12-22 mm

Anchor element outer diameter distal end=20-30 mm

Anchor element outer diameter at base element=22-34 mm

In one embodiment, the device is manufactured having the following dimensions:

Head diameter=50 mm

Head height=19 mm

Anchor length=17 mm

Anchor element inner diameter distal end=24 mm

Anchor element inner diameter at base element=23 mm

Anchor element outer diameter distal end=28 mm

Anchor element outer diameter at base element=30 mm

Fin height at base element=2.5 mm tapering distally

Fin width at base element=2.0 mm tapering distally

### DESCRIPTION OF THE DRAWINGS

FIG. 1 is a representative isometric view of a multi-piece humeral head prosthesis according to the present invention;

FIG. 2 is a representative isometric exploded view of a multi-piece humeral head prosthesis according to the present invention;

FIG. 3 is a representative side cutaway view of a multi-piece humeral head prosthesis according to the present invention;

FIG. 4 is a representative bottom view of a multi-piece humeral head prosthesis according to the present invention;

FIG. 5 is a representative isometric exploded view of a multi-piece humeral head prosthesis according to the present invention;

FIG. 6 is a representative bottom view of a multi-piece humeral head prosthesis according to the present invention;

FIG. 7 is a representative side cutaway view of a single piece humeral head prosthesis according to the present invention;

FIG. 8 is a representative isometric view of a single piece humeral head prosthesis according to the present invention;

FIG. 9 is a representative isometric view of a single piece humeral head prosthesis according to the present invention;

FIG. 10 is a representative side cutaway view a single piece humeral head prosthesis according to the present invention;

FIG. 11 is a representative side view of a humeral head;

FIG. 12 is a representative side view of a humeral head;

FIG. 13 is a representative side view of a multi-piece humeral head prosthesis according to the present invention;

FIG. 14 is a representative side view of a multi-piece humeral head prosthesis according to the present invention;

FIG. 15 is a representative side view of a chassis of a humeral head prosthesis according to the present invention.

### DETAILED DESCRIPTION

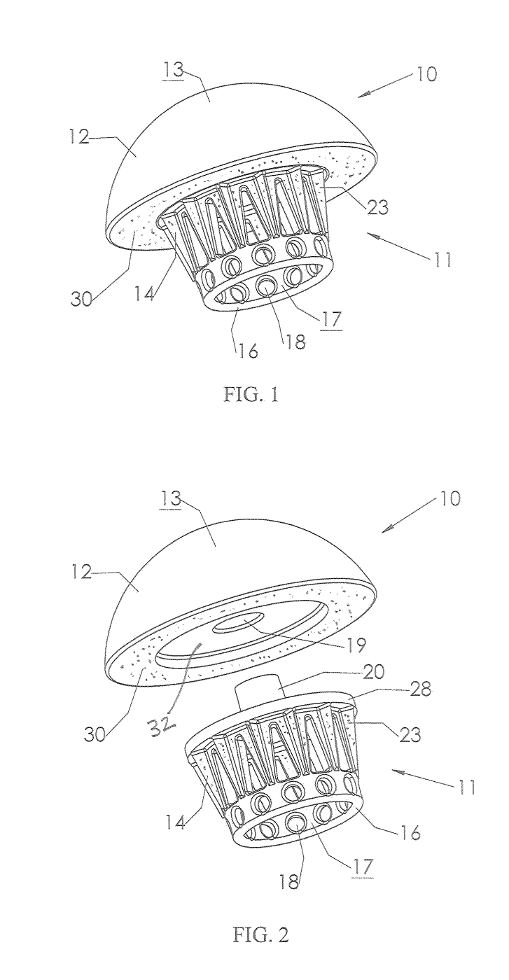

Referring to FIGS. 1 through 4, a preferred embodiment of humeral head prosthesis 10 is shown configured as a two-piece prosthesis. FIGS. 1 through 3 show humeral head prosthesis 10 including chassis 11 and head 12 having articulation surface 13 formed as a spherical segment. Chassis 11 is formed having anchor element 14 that projects distally below head 12. A plurality of suture anchor apertures 18 are formed near distal end 16 of anchor element 14. Suture anchor apertures 18 are configured to permit the passage of a suture according to a method of the invention discussed herein below. Distal end 16 of anchor element 14, and an inner edge defining each of the suture anchor apertures 18 includes a polished surface 17 adapted to prevent surface abrasion between a suture that may be guided through any one of the plurality of suture anchor apertures 18.

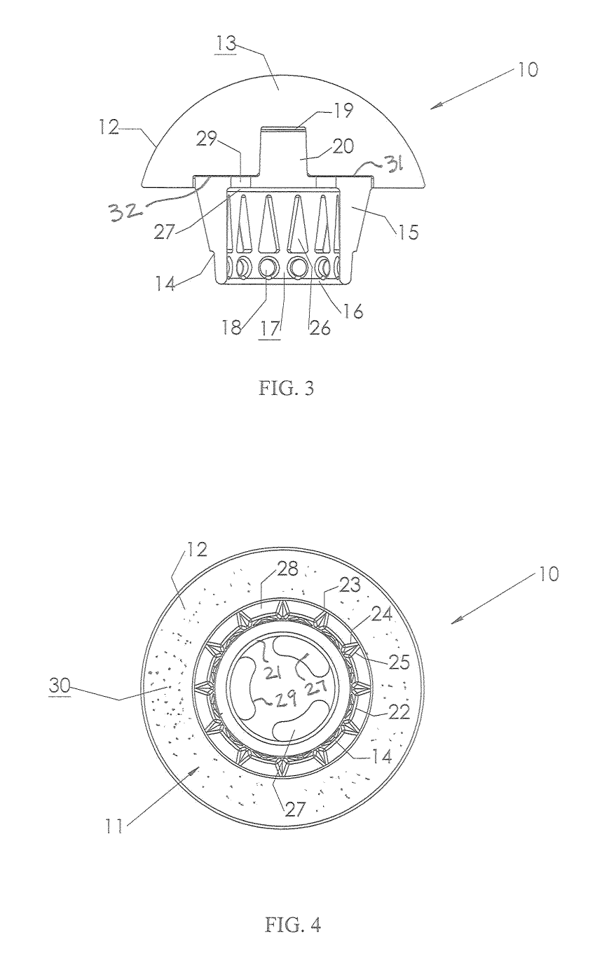

Referring to FIGS. 2 and 3, head 12 includes coupling aperture 19 formed in a distal face 32 of head 12 that cooperates with post 20 that extends from base element 28 to secure head 12 to chassis 11. In the preferred embodiment, coupling aperture 19 and post 20 include cooperating Morse tapers.

Referring to FIGS. 3 and 4, chassis 11 includes anchor element 14 having tapering cross-section 15 that diminishes in thickness as a distance from base element 28 increases, (distally converging). As seen in FIG. 4, inner surface 21 of anchor element 14 is relatively featureless. Outer surface 22 of anchor element 14 includes a plurality of fins 23, each fin 23 comprising a pair of converging wall segments 24 and 25 that taper distally as a distance from head 12 increases, (distally converging). The plurality of fins 23 create a multifaceted outer surface 22 that counters rotation of chassis 11 once impacted into the humeral head. Revascularization apertures 26, shown in FIG. 3 are formed through anchor element 14 from inner surface 21 to outer surface 22 to promote bone growth and revascularization both inside and outside anchor element 14.

FIG. 3 also shows post 20 formed on proximal face 31 of base element 28 inserted into 19 formed in distal face 32 of head 12. Also shown in to FIGS. 3 and 4 are a plurality of blind holes 29. Blind holes 29 are formed in base element 28 of chassis 11. Each blind hole 29 includes a penetrable wall 27 that may be readily pierced or drilled through to accommodate insertion of an osteotome or other tool to assist in removal of chassis 11 from the bone where such removal has become necessary.

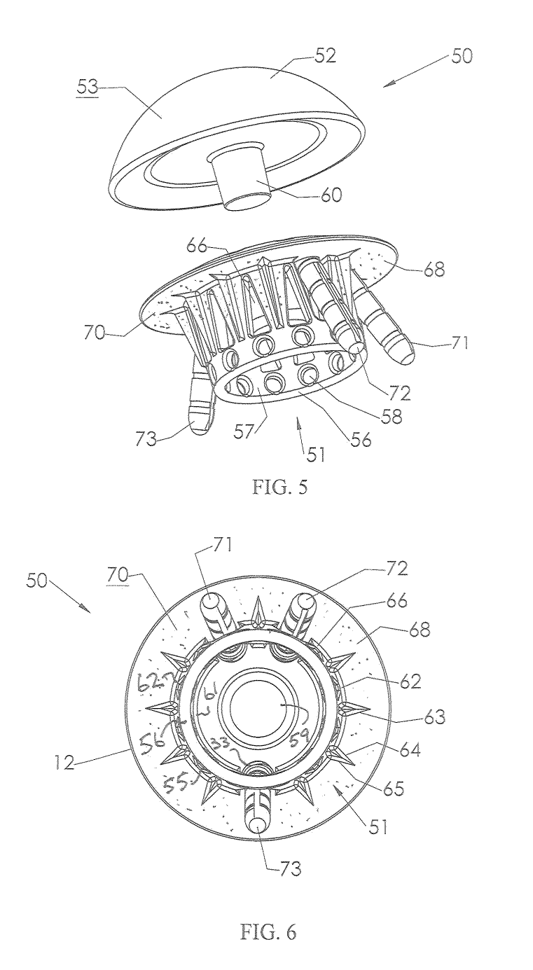

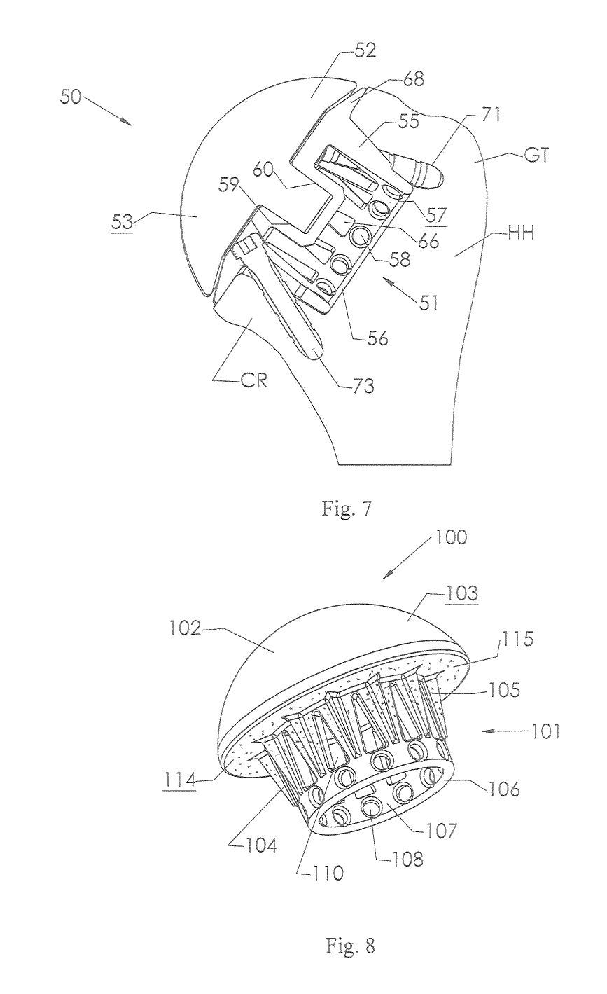

Referring to FIGS. 5 through 7 an alternate embodiment of humeral head prosthesis 50 is shown configured as a two-piece prosthesis. Humeral head prosthesis 50 includes chassis 51 and head 52 that includes articulation surface 53 formed as a spherical segment. Chassis 51 is formed including anchor element 54 that projects distally below head base element 68. A plurality of suture anchor apertures 58 are formed near distal end 56 of anchor element 54. As with the preferred embodiment described above, suture anchor apertures 58 are configured to permit passage of suture according to a method of the invention discussed below. Distal end 56 of anchor element 54, and an inner edge defining each of the suture anchor apertures 58 includes a polished surface 57 adapted to prevent surface abrasion between a suture that may be guided through any one of the plurality of suture anchor apertures 58. As seen in FIG. 5, head 52 includes post 60 that extends proximally from a distal surface of head 52.

Referring to FIG. 6, coupling socket 59 is formed in chassis 51 and extends distally from base element 68. Post 60 and socket 59 have a cooperating fit configured to secure head 52 to chassis 51. FIG. 6 also shows chassis 51 configured having anchor element 54 having tapering cross-section 55 that diminishes in thickness as a distance from base element 68 increases, (distally converging). As seen in FIG. 6, inner surface 61 of anchor element 54 is relatively featureless. Outer surface 62 of anchor element 54 includes a series of fins 63, each fin comprising a pair of converging wall segments 64 and 65 that taper distally as a distance from head 12 increases, (distally converging). Revascularization apertures 66, shown in FIG. 5 are formed through anchor element 54 from inner surface 61 to outer surface 62 to promote bone growth and revascularization both inside and outside anchor element 54.

Referring to FIG. 6 a plurality of blind holes 69 are formed in base element 68 of chassis 51. Each blind hole 69 includes a penetrable wall 67 that may be readily fractured, pierced or drilled through to accommodate insertion of an osteotome or other tool to assist in removal of chassis 51 from the bone where such removal has become necessary.

FIGS. 5 through 7 show bone anchors 71, 72, (shown in FIGS. 5 and 6), and 73 arranged preferably so that on installation of humeral head prosthesis 50, anchors 71 and 72 are oriented so as to be set into the greater tuberosity GT and anchor 73 is oriented so as to be set into the calcar region CR of humeral head HH. As seen in FIG. 6, each of the bone anchors 71, 72 and 73 are adapted for insertion through and threaded engagement with one of the plurality of bone anchor apertures 33 formed through base element 68.

FIG. 8 shows unitary humeral head prosthesis 100 an alternate embodiment that has as one advantage a lowered cost of manufacture as compared to the multi-piece embodiments previously discussed. Humeral head prosthesis 100 includes chassis 101 to which head 102 is integrally formed. Head 102 includes articulation surface 103 formed as a spherical segment. Chassis 101 includes anchor element 104 which extends from base element 115. Anchor element 104 includes a plurality of distally converging fins 105. A plurality of revascularization apertures 110 are formed one between each of the plurality of distally converging fins 105. Distal end 106 of anchor element 104 includes polished surface 107. A plurality of suture anchor apertures 108 are formed proximate to distal end 106. As shown in FIG. 8, the face of base element 115 and a substantial portion of anchor element 104 include textured surface 114.

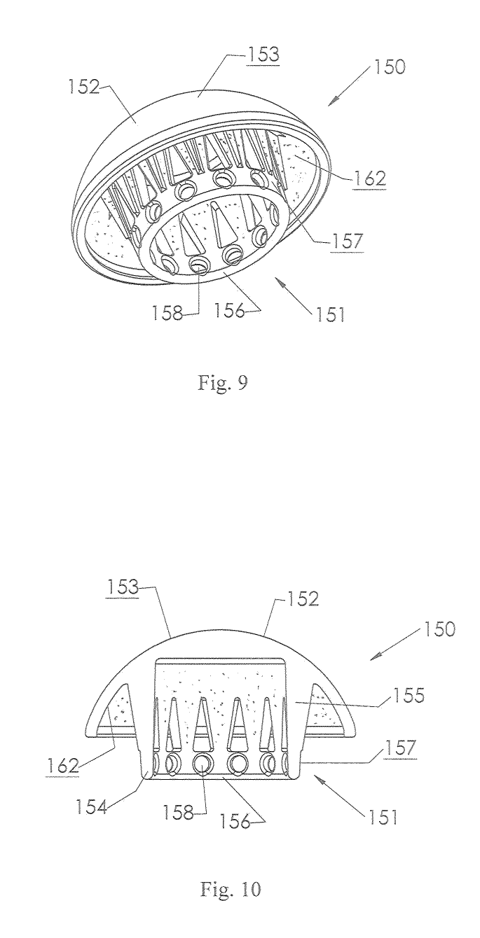

FIGS. 9 and 10 show an alternate embodiment of humeral head resurfacing prosthesis 150 configured as a unitary prosthesis use where resurfacing of the humeral head is possible and indicated. Humeral head resurfacing prosthesis 150 includes chassis 151 to which head 152 is integrally formed. Head 152 includes articulation surface 153 formed as a spherical segment. Chassis 151 includes anchor element 154 which extends from base element 161. Anchor element 154 includes a plurality of distally converging fins 163. A plurality of revascularization apertures 160 are formed one between each of the plurality of distally converging fins 155. Distal end 156 of anchor element 154 includes polished surface 157. A plurality of suture anchor apertures 158 are formed proximate to distal end 156. As seen in FIG. 9, anchor element 154 includes tapering cross-section 155. As shown in FIGS. 9 and 10, head 152 includes partially concave surface 162 which is configured to cooperate with a prepared surface of the humeral head to be resurfaced. Concave surface 162 and a substantial portion of anchor element 154 include textured surface 164.

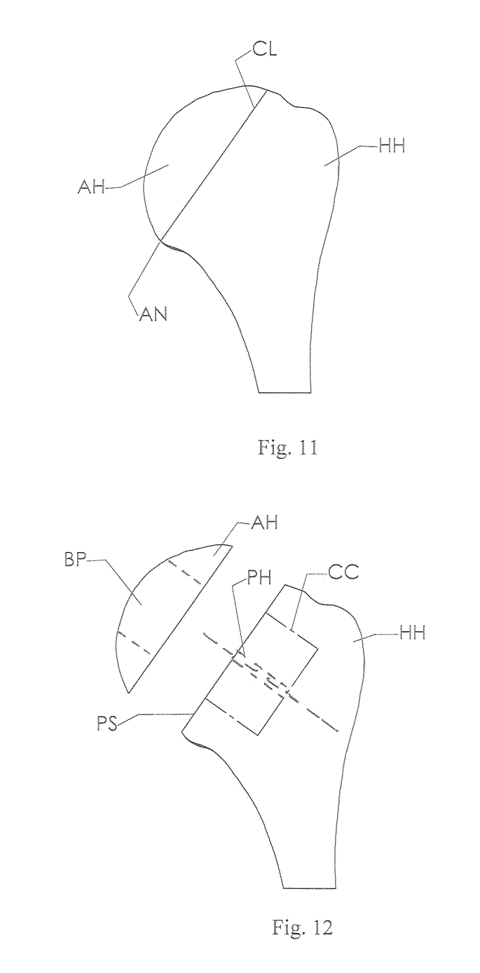

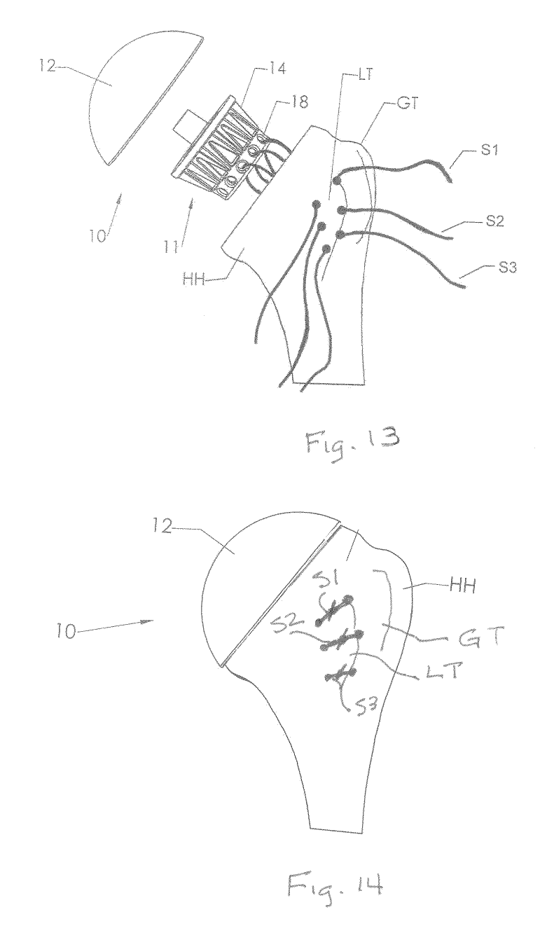

Referring to FIG. 11 and according to a method of the present invention, humeral head HH is prepared for installation of humeral head prosthesis 10 shown in FIGS. 1 through 4 by first removing anatomical head AH along cutline CL defining a plane that corresponds generally to anatomical neck AN. FIG. 12 shows the next steps in the preparation including the drilling of pilot hole PH that lies substantially perpendicular to planar surface PS. Pilot hole PH is used to guide a hole saw, (not shown), for cutting circular cut CC having a diameter that is substantially equal to a diameter of distal end 16 of humeral head prosthesis 10 shown in FIG. 1. Also according to one methodology of the present invention bony plug BP may be harvested from anatomical head AH by means of a hole saw having a diameter substantially equal to an inside diameter of anchor element 14 of humeral head prosthesis 10 shown in FIG. 1. Bony plug BP may be used as bone graft material that is inserted into the interior of anchor element 14 in cases where there has been deterioration of the trabecular bone at the center of humeral head HH.

Referring to FIG. 15, in the event that removal of humeral head prosthesis 10 is required, the surgeon removes head 12, shown in FIG. 2 and hole saw HS having a diameter slightly greater than a diameter of base element 28 is used to form removal cut RC. Chassis 11 is then worked free of underlying bone and removed together with bony head segment HS.

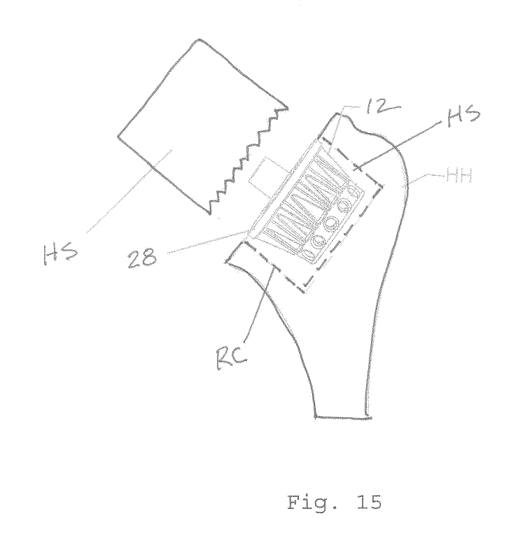

Referring to FIGS. 13 and 14, sutures S1, S2 and S3 are passed through first, second and third tunnels T1, T2 and T3 formed through the cortical bone and terminating in circular cut CC. Distal end 16 of chassis 11 is then pressed into circular cut CC. Referring to FIGS. 3 and 4, tapering cross-section 15 creates a press fit as anchor member 14 is pressed into circular cut CC. Additionally, fins 23 counter rotation of chassis 11 once positioned in circular cut CC. As anchor member 14 is pressed further into circular cut CC, sutures S1, S2 and S3 are pulled tight and as shown in FIG. 14 knotted across lesser tuberosity LT to secure the subscapularis tendon and/or the lesser tuberosity against humeral head HH.

The description of the illustrated embodiments has been presented for purposes of illustration and description and is not intended to be exhaustive or to limit the invention to the precise form or to exemplary embodiment(s) and implementation(s) disclosed. Modifications and variations will be apparent to practitioners skilled in this art. Process steps described might be interchangeable with other steps in order to achieve the same result. At least one preferred embodiment was chosen and described in order to best explain the principles of the invention and a best mode of practical application, thereby to enable others skilled in the art to understand the invention and the various modifications that are suited to the particular use or implementation contemplated. The scope of the invention is defined by the claims appended hereto and their equivalents. Reference to an element in the singular is not intended to mean “one and only one” unless explicitly so stated, but rather means “one or more.” No claim element herein is to be construed under the provisions of 35 U.S.C. Sec. 112, sixth paragraph unless the element is expressly recited using the phrase “means for . . . ”

What is claimed is:

1. A humeral head prosthetic device comprising:
a chassis including a base element;
a head attached to the chassis, the head including an articulation surface; and
a tapered and multifaceted anchor element attached to and projecting distally from the base element, the tapered and multifaceted anchor element including a plurality of revascularization apertures formed through a wall of the tapered and multifaceted anchor element.

2. The humeral head prosthetic device of claim 1 further comprising:
a textured surface formed on a distal face of the base element;
a textured surface formed on an inner surface of the tapered and multifaceted anchor element; and
a textured surface formed on an outer surface of the tapered and multifaceted anchor element.

3. The humeral head prosthetic device of claim 1 further comprising:
a plurality of bone anchors apertures formed through the base element; and
a plurality of bone anchors adapted for insertion through the bone anchor apertures, the plurality of bone anchors further adapted for threaded engagement with the base element.

4. The humeral head prosthetic device of claim 1 further comprising:
a plurality of suture anchor apertures formed proximally to a distal end of the tapered and multifaceted anchor element; and
the distal end of the tapered and multifaceted anchor element that surrounds each of the plurality of suture anchor apertures including a polished surface.

5. The humeral head prosthetic device of claim 1 wherein the anchor element further comprises a plurality of blind holes formed in the base element, each of the plurality of blind holes including a penetrable wall configured to be readily penetrated.

6. The humeral head prosthetic device of claim 1 further comprising:
the head removably attachable to the chassis;
a post having a tapered sidewall, the post formed on a proximal face of the chassis; and
a socket formed in a distal face of the head, the socket having a tapered sidewall adapted to cooperate with the tapered sidewall of the post, the post and socket adapted for removably attaching the head to the chassis.

7. The humeral head prosthetic device of claim 1 further comprising:
the head removably attachable to the chassis;
a post having a tapered sidewall, the post formed on a distal face of the head; and
a socket formed in a proximal face of the chassis, the socket having a tapered sidewall adapted to cooperate with the tapered sidewall of the post, the post and socket adapted for removably attaching the head to the chassis.

8. The humeral head prosthetic device of claim 1 wherein the head further comprises a partially concave distal surface formed on a distal face of the head.

9. A humeral head prosthetic device comprising:
a chassis including a base element;
a head attached to the chassis, the head including an articulation surface; and
a tapered and multifaceted anchor element attached to and projecting distally from the base element, the tapered and multifaceted anchor element including a plurality of revascularization apertures formed through a wall of the tapered and multifaceted anchor element; and
one or more of blind holes formed in the base element, each of the plurality of blind holes including a penetrable wall configured to be readily penetrated.

10. The humeral head prosthetic device of claim 9 further comprising:
a textured surface formed on a distal face of the base element;
a textured surface formed on an inner surface of the anchor element; and
a textured surface formed on an outer surface of the tapered and multifaceted anchor element.

11. The humeral head prosthetic device of claim 9 further comprising:
a plurality of bone anchors apertures formed through the base element; and
a plurality of bone anchors adapted for insertion through the bone anchor apertures, the plurality of bone anchors further adapted for threaded engagement with the base element.

12. The humeral head prosthetic device of claim 9 further comprising:
a plurality of suture anchor apertures formed proximally to a distal end of the tapered and multifaceted anchor element; and
the distal end of the tapered and multifaceted anchor element that surrounds each of the plurality of suture anchor apertures including a polished surface.

13. The humeral head prosthetic device of claim 9 further comprising:
the head removably attachable to the chassis;
a post having a tapered sidewall, the post formed on a proximal face of the chassis; and
a socket formed in a distal face of the head, the socket having a tapered sidewall adapted to cooperate with the tapered sidewall of the post, the post and socket adapted for removably attaching the head to the chassis.

14. The humeral head prosthetic device of claim 9 further comprising:
the head removably attachable to the chassis;
a post having a tapered sidewall, the post formed on a distal face of the head; and
a socket formed in a proximal face of the chassis, the socket having a tapered sidewall adapted to cooperate with the tapered sidewall of the post, the post and socket adapted for removably attaching the head to the chassis.

15. The humeral head prosthetic device of claim 9 wherein the head further comprises a partially concave surface formed on a distal face of the head.

16. A humeral head prosthetic device comprising:
a chassis including a base element;
a head removably attachable to the chassis;
means disposed between the head and the chassis for removably attaching the head to the chassis; and
a tapered and multifaceted anchor element attached to and projecting distally from the base element, the tapered and multifaceted anchor element including a plurality of revascularization apertures formed through a wall of the tapered and multifaceted anchor element.

17. The humeral head prosthetic device of claim 16 wherein the means disposed between the head and the chassis for removably attaching the head to the chassis further comprises:
a post having a tapered sidewall, the post formed on a proximal face of the chassis; and
a socket formed in a distal face of the head, the socket having a tapered sidewall adapted to cooperate with the tapered sidewall of the post, the post and socket adapted for removably attaching the head to the chassis.

18. The humeral head prosthetic device of claim 16 wherein the means disposed between the head and the chassis for removably attaching the head to the chassis further comprises:
a post having a tapered sidewall, the post formed in a distal face of the head; and
a socket formed the in proximal face of the chassis, the socket having a tapered sidewall adapted to cooperate with the tapered sidewall of the post, the post and socket adapted for removably attaching the head to the chassis.

19. The humeral head prosthetic device of claim 16 further comprising:
a textured surface formed on a distal face of the base element;
a textured surface formed on an inner surface of the tapered and multifaceted anchor element; and
a textured surface formed on an outer surface of the tapered and multifaceted anchor element.

20. The humeral head prosthetic device of claim 16 further comprising:
a plurality of suture anchor apertures formed proximally to a distal end of the tapered and multifaceted anchor element; and
the distal end of the tapered and multifaceted anchor element that surrounds each of the plurality of suture anchor apertures including a polished surface.

21. The humeral head prosthetic device of claim 16 further comprising:
a plurality of bone anchors apertures formed through the base element; and
a plurality of bone anchors adapted for insertion through the bone anchor apertures, the plurality of bone anchors further adapted for threaded engagement with the base element.

22. The humeral head prosthetic device of claim 16 wherein the anchor element further comprises one or more blind holes formed in the base element of the anchor element, each of the one or more blind holes including a penetrable wall configured to be readily penetrated.

23. An implantable prosthesis comprising a plurality of blind holes formed in a surface of the prosthesis, each of the plurality of blind holes including a penetrable wall configured so as to be readily penetrated to accommodate insertion of a tool for aiding in removal of the implantable prosthetic device.
